(19) United States
(12) Patent Application Publication
(10) Pub. No.: US 2021/0192919 A1
(43) Pub. Date: Jun. 24, 2021

(54) A MEDICAL ALARM RELAY DEVICE FOR THE INCAPACITATED

(71) Applicant: John Graeme Hookway, Auckland (NZ)

(72) Inventor: John Graeme Hookway, Auckland (NZ)

(21) Appl. No.: 17/257,583

(22) PCT Filed: Jul. 29, 2019
(86) PCT No.: PCT/NZ2019/050088

§ 371 Date: Jan. 3, 2021

(30) Foreign Application Priority Data
Aug. 6, 2018 (NZ) 744977

(51) Int. Cl. G08B 21/04; A61B 5/00; A61B 5/11; G16H 40/67

(52) U.S. Cl. CPC G08B 21/043; A61B 5/0077; A61B 5/0022; A61B 2562/0204; A61B 5/1118; G08B 21/0453; G16H 40/67; A61B 5/1112

(57) ABSTRACT

The invention relates to the technical field of wearable emergency equipment, in particular a medical alarm relay device for the incapacitated. This comprises an automatic programmable interface (API) for checking the device, a communication network for interconnecting an automatic programmable interface (API) from a server to a device & auxiliary device, and a reliability circuit. The invention provides system relays by cooperating with the automatic programmable interface (API), communication network and reliability circuit. The reliability circuit provides a system relay, reliably monitors user's activities and the device itself. User-device interactions and the device itself alert invalid events and lack of user or equipment response will trigger an alarm to others in time to rescue users.

### FIELD

The present invention relates to the technical field of wearable emergency equipment, in particular to a medical alarm relay device for the incapacitated.

### BACKGROUND

The idea of wearable emergency equipment is not novel, and patent applications have been published describing, for example, the use of various communication devices; e.g., the use of telephones, pagers or dedicated devices for transmitting call-and-rescue signals. However, users may be physically or medically incapacitated and unable to operate the device.

In addition, there is an unavoidable problem in letting people seek help on their own. Some people are unable to evaluate their condition and illness, which leads to a lack of action and willpower to seek help. Some people don't seek help and wait for the condition to get worse before deciding the disease is severe, but it may be too late.

By using a “fall detector”, these problems can be partially solved. When wearable devices detect that the user has fallen, the “fall detector” automatically sends out a help signal. However, this applies only to selected emergencies, and triggering an alarm requires action first. For example, it does not apply to people lying unconscious in bed.

At the same time, there is a problem with such devices: they may fail due to negligence. Batteries on a cell phone or any device may die, or for other reasons, the device may stop working. The key is to ensure the “fail-safe” operation of the device and user-device interactions, even in the event of failure, no harm will be caused or injury will be minimized.

Therefore, in view of the above situation, there is an urgent need to develop a medical alarm relay device for the the incapacitated to overcome the shortcomings of the current practical application.

### SUMMARY

The invention aims to provide a medical alarm relay device for the incapacitated to solve the problems mentioned in the background.

To achieve the above purpose, the invention provides the following technical scheme:

A medical alarm relay device for the incapacitated, including an automatic programmable interface (API) for checking the device, a communication network and for interconnecting an automatic programmable interface (API) from a server to equipment & auxiliary equipment, and a reliability circuit.

As a further scheme of the invention, the device comprises of:

a) The countdown display on the device, which is a countdown timer for warning the user that the device will transmit emergency signals if no action is taken;

b) A method of sending a signal indicating a medical emergency from unresponsive users;

c) An automatic programmable interface (API) operated from a remote database server is used to send signals when the device is not responding.

d) The method by which a user performs specific actions on a device to indicate the transmission of signals in medical emergencies;

e) Wireless transmitters that use wireless or mobile phone services to transmit signals over the Internet;

f) A method for transmitting information, which comprises the heart rate of the user (31), GPS position data and medical information;

g) Shock-proof and water-proof bracelets or watches;

h) An optional feature of the device (33) which provides a telephone device for direct voice communication with a user

As a further scheme of the invention, the communication network comprises:

a) Communication between the device and the server;

b) The communication between the server and the auxiliary device, when the device does not respond;

c) The communication between the server and the receiver, when the device does not respond;

d) The server transmits complete and orderly signals from the device to the receiver through text, e-mail and voice messages, with short message information, hyperlinks, attachments, audit logs, maps and heart rate charts.

e) A list of receivers, which comprises one or more electronic addresses and communication devices between receivers and the server.

As a further scheme of the invention, the automatic programmable interface (API) comprises the following:

a) Based on the input mechanism of network data, users can use the displayed information to update the automatic programmable interface (API).

b) Audit logs of the vital signs and activities of the user and audit logs of the device activities;

c) Information collected from the device, such as user activity, device activity, heartbeat and GPS location information on the device that detect and provide alarms for vital signs that exceed the preset parameters;

d) An automatic reasoning task that determines the communication needs of the device status receivers or user status receivers, and informs the device or the user of any inadequate communication performance.

e) Communications transmitted to the recipient and the user.

As a further scheme of the invention, the method of the reliability circuit comprises the following redundancies:

a) The use of multiple servers;

b) Multiple signals (including short message, e-mail and voice mail) are sent by various communication methods;

c) Multiple signals are transmitted through multiple bandwidth channels;

d) Multiple signals are transmitted through multiple telecommunication providers;

e) Multiple signals are sent to multiple receivers;

f) Match the device, the user and the transmission signal with the expected value of the system.

g) Communication with the user through the equipment and the auxiliary equipment;

h) Notifies the user (31) and the receiver (49) when the device (33) settings are changed;

i) Continuous and periodic checks are performed through the automatic programmable interface (API).

Compared with the existing technology, the beneficial effect of the invention is that the invention provides system relays by cooperating with an automatic programmable interface (API), communication network and reliability circuit. Reliability circuit provides a system relay, reliably monitors user activities and devices, user-device interactions, and the device itself alert invalid events and lack of user or equipment response will trigger an alarm to others in time to rescue users.

### BRIEF DESCRIPTION OF THE DRAWINGS

FIG. 1 is a medical alarm device in the existing technology.

FIG. 2 is a schematic diagram of a signal sent by an unresponsive user in a medical alarm relay device for the incapacitated.

FIG. 3 is a schematic diagram of a medical alarm device relay failure in which the user does respond and no signal is sent.

FIG. 4 is a schematic diagram of the automatic programmable interface (API) in the medical alarm relay device for the incapacitated.

FIG. 5 is a schematic diagram of signal transmission among devices, servers and receivers in medical alarm relay device for the incapacitated.

FIG. 6 is the working flow chart of medical alarm relay device for the incapacitated.

In figures: 31—user, 32—button, 33—device, 34—display timer, 35—antenna, 36—signal, 41—automatic program, 42—wireless communication, 46—communication, 47—auxiliary device, 48—send, 49—receiver, 51—server 51, 52—re-send, 56—heart rate information, 57—GPS location, 58—audit log, 59—medical information.

### DESCRIPTION OF EMBODIMENTS

The technical scheme of the patent is described in further detail in connection with specific embodiments.

Embodiments of the patent are described in detail as following, and examples of the embodiments are shown in the drawings in which identical or similar labels throughout represent identical or similar elements or elements with the same or similar functions. The following embodiments described by reference to the accompanying drawings are illustrative and are used only to explain the patent, not to constrain it.

In the description of this patent, it should be noted that unless otherwise specified and limited, the terms “connection” and “setting” should be understood in a broad sense. For example, they may be fixed connection, setting, or removable connection, setting, or integrated connection and setting. For those skilled in the art, the specific meaning of the above terms in this patent may be understood according to the specific circumstances.

### Embodiment 1

A medical alarm relay device for the incapacitated, comprises:

A) An Automatic Programmable Interface (API) for checking device 33;

B) A Communication network: API is interconnected from server 51 to device 33 and auxiliary device 47;

C) A Reliability circuit.

A: Device 33

The device 33 is used in conjunction with an automatic programmable interface (API), communication network and a reliability circuit. Reliability circuit provides a system relay, which can reliably monitor user activities and equipment 33 itself.

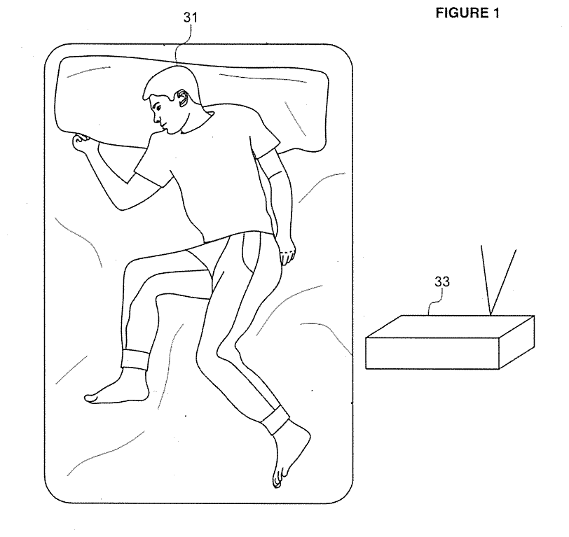

Referring to FIG. 1, FIG. 1 shows the problems that need to be solved by the medical alarm device 33 in the existing technology. The user 31 does not respond, does not send a signal, the user 31 is in a non-responding state, does not press the activation device 33 button 32, nor does it send the device 33 signal.

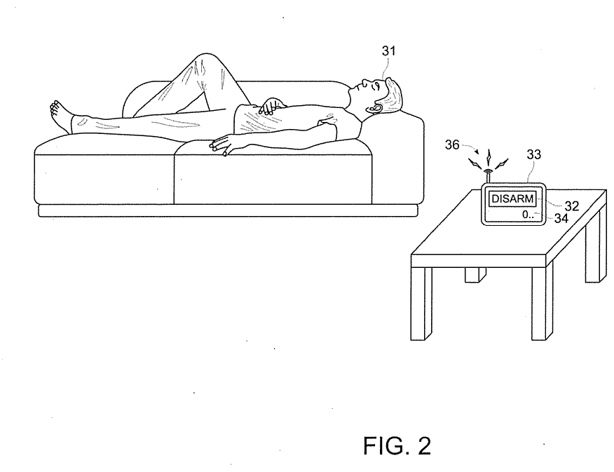

Referring to FIG. 2, FIG. 2 shows a device 33 that sends signals because the user does not respond. In the attached figure, the user 31 is unable to move, is lying down, and does not press a button 32 to release the device 33 at the end of the display timer 34. Therefore, after the display timer 34 finishes the timing, the signal, 36 is sent out.

If the user does not cancel the alarm within a given warning time, the device 33 will send a signal at a pre-set time. If the display timer 34 time has arrived, the device 33 will give a visual and auditory warning. If the user 31 fails to confirm the warning, the device 33 will send a signal.

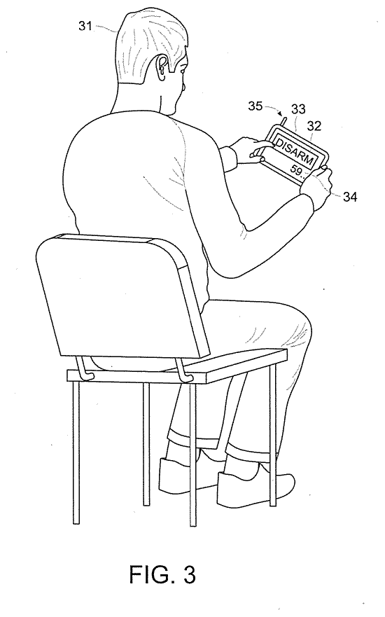

FIG. 3 shows that user 31 releases the device 33 so that the device 33 does not send a signal. In the figure, the user 31 presses the button 32 on the device 33 before the display timer 34 ends, and does not send a signal after pressing the button 32.

As a preferred embodiment of the present embodiment, the device 33 is wearable or mobile and has an independent wireless Internet facility comprising:

a) A display timer 34, with countdown warning;

b) A simple way to easily relieve the emergency transmission of the watch by sliding or pressing the button once or twice;

c) A method for the user 31 to send signals manually.

d) A method for information related to user health to be sent.

e) An optional telephone device for direct voice communication with users and a wireless transmitter capable of sending wireless alarms.

The device 33 comprises the following contents:

a) Microphone

b) Video camera

c) Transceiver

d) Screen

e) Speaker

f) Power source

g) Heart rate sensor

h) GPS unit

i) Memory Storage

j) Logic Board/CPU

k) Memory (RAM&ROM)

I) SIM card

m) Antenna

n) Buzzer/Alarm

The precise circuit and functional relationships between components of the device 33 can undergo many modifications and changes, which are obvious to ordinary technicians in the field.

B: Communication Network

Referring to FIG. 5. The device 33 signals are sent to a receiver(s) 49 through a server 51, information is sent from the device 33 to a server 51, and re-sent 52 to multiple receivers 49. This information comprises heart rate information 56, GPS location 57, audit log 58 and medical information 59.

The communication network comprises:

a) Device 33

b) Auxiliary device 47;

c) Server 51: A computer for storing and sending data over the Internet;

d) Setting dashboard (Internet Web application);

e) Internet, telephone and short message systems;

f) Receiver: Message receiver and its electronic address;

g) API: Automatic programmable interface running in the background;

Finally, message or signal transmission:

a) From transmitter to server 51;

b) From server 51 to receiver 49;

c) From Server 51 to Auxiliary Device 47 (when Device 33 does not respond);

API monitoring information travels between server 51 and device 33.

More detailed explanation of communication networks

a) Device 33: A device 33 sends information to a server 51 and indicates the response status; and sends a signal when the user 31 does not respond;

b) Auxiliary device 47: If a device 33 does not work, the API will communicate the problem to the user, 31 in the auxiliary communication channel, for example, through the voice message of the mobile phone, explaining that the device 33 does not respond;

c) Server 51 stores information from the API and sends GPS location data, heartbeat rate and other information to recipient 49 together with pre-loaded medical information. Server 51 provides load balancing to ensure access to backup server 51;

d) The device 33 is used in conjunction with the dashboard to configure the device 33 and its transmission. The portal for configuration comprises a device for configuration;

i) Receiver's electronic address;

ii) Information packages attached to emergency signals;

iii) The characteristics of device 33 signal deployment, such as the frequency of signal transmission, warning time, etc.

e) Internet, Telephone and Short Message Systems: These are commonly available Tele-communications services that can transmit signals and messages.

f) Receiver-Message Receiver and Its Communication Address, E-mail Address, Short Message and Telephone Number;

C: Automatic Programmable Interface (API)

All components of communication network are interconnected with an API (automatic programmable interface). The API runs in the background and is programmed using explicit specifications, algorithms for automatic reasoning tasks, or a set of rules. For example, when to take action by sending a message.

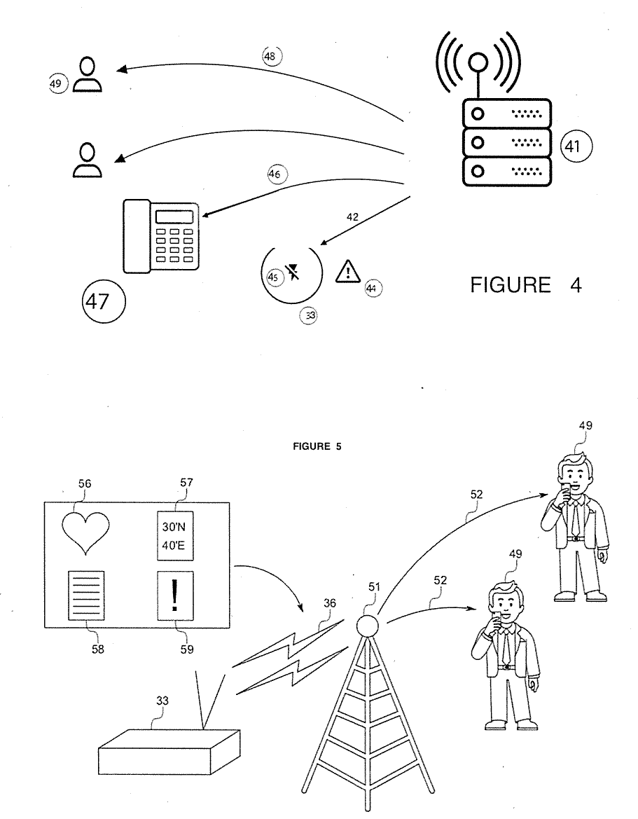

Referring to FIG. 4, which shows an automatic program 41 that communicates with the device 33 wireless communicator 42 to check whether the device 33 responds. In this case, due to hardware and memory failures, the device 33 does not respond. The automatic program will communicate 46 with the auxiliary device 47. If the auxiliary device 33 does not respond, it sends 48 messages to multiple receivers 49.

Essentially, the functions of automatic programmable interface (API) are:

a) Logging in and exiting the device 33 and confirm that it is responsive;

b) Collecting information from the device 33, such as user activity on the device 33, device 33 activity, heartbeat and GPS location information. API detects and warns when vital signs exceed the preset parameters;

c) Creating and storing audit logs of activities;

d) Cooperating with the program system based on intelligent rules to perform automatic reasoning tasks to follow up communication. API can communicate with a user's personal digital assistant;

e) Providing access to the portal site, which comprises settings panels for setting user preferences, such as the time and attributes of the alert to be disabled, who to contact and how to contact when signals and medical information are received.

The best way to monitor an automatic programmable interface (API):

The best way to use the automatic programmable interface (API) system is to reduce all possible data to a single aggregate metric using data received by multiple sensors based on the device 33, and implement automatic reasoning algorithms. Emergency levels are calculated from multiple metrics to provide an automatic programmable interface (API) personal monitoring. Aggregation metrics are displayed on color-coded logarithmic scales with alerts.

D: Reliability Circuit

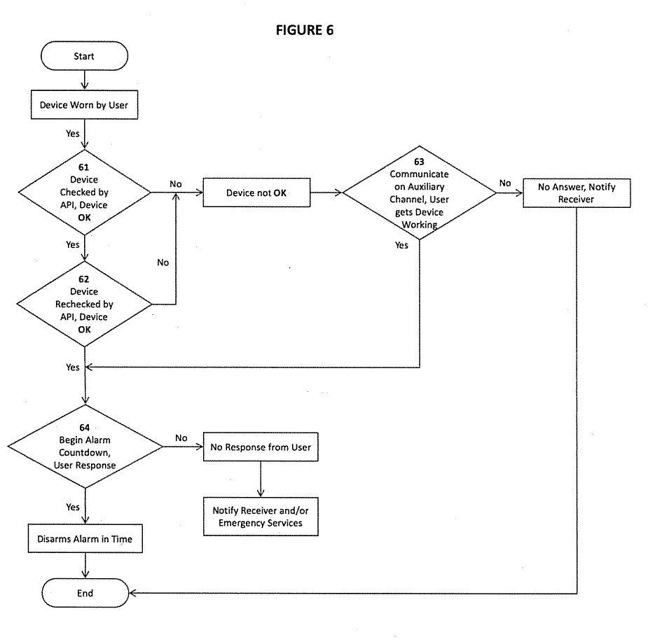

Referring to FIG. 6, which is a flowchart illustrating the approximate steps in a communication network data stream. The flow chart shows that the core item of automatic reasoning is the diamond decision-making box. The box shows that the device 33 is checked through the automatic programmable interface (API) to ensure that the device 33 is in good working condition. This is a continuous operation, if the device 33 is abnormal, then re-check. If the device 33 cannot respond, the user 31 is notified on the auxiliary channel (telephone). If the user 31 does not respond, the receiver 49 will be notified. Otherwise, the display timer 34 starts timing and notifies the receiver 49 If the user 31 does not respond.

To ensure reliability, multiple parallel channels are used by using redundancy when feasible. For example, multiple device signals 36, multiple servers 51, communication with multiple receivers 49 over multiple communication channels (short message, telephone and e-mail), and so on.

### Embodiment 2

The difference between the present implementation and the first embodiment is that the other reliability measures component, the Continuous Improvement System, comprises the following:

a) Expected behavior is modeled based on actual behavior (for example, the reconfigured warning time input to the dashboard is compared with the actual warning time, etc.);

b) The auxiliary communication device 33 is used to communicate any fault to the user 31 and optionally to the receiver 49;

c) When the device 33 settings are changed, assistance notifications will be sent to the user 31 and some receivers 49;

d) The Automatic Programmable Interface (API) periodically inspects device 33 to provide an early warning system.

The above is only the preferred implementation of the invention. It should be pointed out that for the technical personnel in the field, some variation and improvement can be made without departing from the concept of the invention. These should also be considered as the protection scope of the invention, which will not affect the effect of the implementation of the invention and the practicability of the patent.

I claim:

1. A method for sending an alert, comprising:
Connecting a smartwatch to a database on a server via an application program interface (API) to detect the smartwatch operability;
periodically or continuously detecting a GPS location and a heart rate of the user using the smartwatch;
a user setting a pre-set time into the smartwatch, wherein said pre-set time indicating when said user expects to be awake and responsive;
the API transferring the pre-set time via a wireless module on the smartwatch to the database;
detecting on the smartwatch, an interaction between the user and the smartwatch at the pre-set time and the API transferring details of the interaction to the database;
periodically or continuously detecting a GPS location and a heart rate of the user using the smartwatch;
the API transferring the GPS location and a heart rate of the user to the database;
Wherein the improvement comprises an non-Biometric user monitoring system that includes:
Wherein when the server detects a disconnection between the smartwatch and the API the server initiates sending an alert to one or more receivers;
Wherein the lucidity and state of awareness of the user is tested at the pre-set time by detecting the interaction between the user and the smartwatch;
Wherein in response to the server detecting an absence of interaction between the user and the smartwatch at the pre-set time, the server initiates sending an alert to the one or more receivers;
Wherein the user can interrupt/cancel transmittal of an alert to the one or more receivers by a predetermined signal.

2. The method according to claim 1, wherein detecting the interaction between the user and the smartwatch at the pre-set time follows from the smartwatch's processor registering physically and mentally capable user actions on the smartwatch interface.

3. The method according to claim 1, wherein the smartwatch periodically transmits via the wireless module and the API and thereby updating the database with any changes to the pre-set time.

4. The method according to claim 1, wherein the pre-set time comprising: a time of day and a specific time interval.

5. The method according to claim 1, wherein the user setting a modality for interrupting and cancelling the transmittal of the alert to the one or more receivers via the smartwatch.

6. The method according to claim 1, wherein the user's interaction with the smartwatch comprising: sliding or pressing a button once or twice on said smartwatch.

7. The method according to claim 1, wherein an audit log stores the user interaction data, the heart rate data and the GPS position data.

8. The method according to claim 1, wherein the alert to the one or more receivers includes sending the GPS position data of the smartwatch and heart rate data of the user to the said receivers.

9. The method according to claim 1, wherein interrupting and cancelling the transmittal of alert to the one or more receivers and transferring the pre-set time from the smartwatch to a database on a server via the API comprises:
Inputting data onto the smartwatch that connects through the API to the server;
Inputting data onto a cloud-based user portal that connects through the API to the server.

10. The method according to claim 1, wherein the one or more receivers comprise one or more electronic addresses and communication devices.

11. The method claimed in claim 1, wherein transmissions between the smartwatch and the one or more receivers via the API, database and server includes a reliability circuit comprising:
a) An intelligent automatic transfer switch mechanism that detects disruptions to any components in the circuit and switches data flow and signal transmission to alternative redundant components in the circuit;
b) Physical network arrangements with components operating in parallel circuit configuration to provide redundancy comprising:
i) The use of multiple servers (51); and load balancers;
ii) Communication with the user through the smartwatch (33) and a mobile phone (47);
d) Signal transmissions operating in parallel circuit configuration to provide redundancy comprising:
i) Multiple copies of signals including short message, e-mail and voice mail are sent by various communication methods;
ii) Multiple copies of signals are transmitted through multiple bandwidth channels;
iii) Multiple copies of signals are transmitted through multiple telecommunication providers;
iv) Multiple copies of signals are sent to one or more receivers (49);
e) Processing operations in parallel circuit configuration to provide redundancy comprising:
i) Data matching operations of the device (33) the user (31) and signal transmission to detect inconsistent values in the system;
ii) Continuous and periodic data audit checks performed through the automatic programmable interface (API).

12. An apparatus for sending an alert, comprising:
A cloud-based server that comprises:
a database,
a scheduler and
an application programming interface (API);
one or more receivers comprises email and telephone numbers for text messages;
A smartwatch comprises: one or more user interface controls, a wireless module,
a GPS module,
a heart rate sensor, an audit log;
Detecting user interaction with at least one of the one or more user interface controls, wherein said user interface controls are visual display interface and buttons on the smartwatch, and controlling the smartwatch based on the detected user interaction;
Wherein the GPS module provides GPS position data of the smartwatch, and a heart rate sensor provides heart rate data of a user;
Wherein the audit log stores the user interaction data, the heart rate data of the user and the GPS position data of the smartwatch;
Wherein the smartwatch visual display interface includes a means for entering a time of day indicating when the user expects to be awake and responsive; and a specific time interval and storing said time values in the audit log;
Wherein the data in the audit log is transmitted via the wireless module and the API to the database;
The smartwatch periodically transmits via the wireless module and the API and thereby updates the database with any changes to the pre-set time;
Wherein the server transmits an emergency alert to the one or more receivers at the pre-set time that is stored in the database, said emergency alert comprises GPS position data of the smartwatch and the heart rate data of the user;
Wherein a specific interaction of the user with the smartwatch during the specific time interval cancels the transmission of the emergency alert to the one or more receivers and thereby signals that the user is responsive;
Wherein the API checks for a transmission from the smartwatch at times dictated by the scheduler;
Wherein the server hosts scheduled tasks configured to initiate the transmission of an alert sent from the server to the receivers when there is an absence of transmission from the smartwatch at times dictated by the scheduler and thereby signal the inoperability of the smartwatch; and
Wherein the improvement comprises the transmission of alerts to the one or more receivers independently of the biometric status or interaction of the user.

13. The apparatus claimed in claim 12, wherein a visual display interface for data input on a cloud-based user portal that connects to the database can receive the time of day and the specific time interval and store the said data into the database.
